(19) United States
(12) Patent Application Publication
(10) Pub. No.: US 2009/0136514 A1
(43) Pub. Date: May 28, 2009

(54) Tetracyclines for Treating Ocular Diseases and Disorders

(76) Inventor: David F. Power, Trabuco Canyon, CA (US)

Correspondence Address: ROPES & GRAY LLP, PATENT DOCKETING 39/361, 1211 AVENUE OF THE AMERICAS, NEW YORK, NY, 10036-8704, US

(21) Appl. No.: 12/086,146

(22) PCT Filed: Dec. 7, 2006
(86) PCT No.: PCT/US2006/047174

§ 371 Date: Jun. 6, 2008

Related U.S. Application Data
(60) Provisional application No. 60/748,506, filed on Dec. 7, 2005.

(51) Int. Cl. A61K 39/395; A61K 31/65

(52) U.S. Cl. 424/145.1; 514/154

(57) ABSTRACT

Methods and compositions are disclosed for treating a patient suffering from a condition associated with a retinal and/or choroidal disease or disorder of the eye involving endothelial cell dysfunction, especially vascular endothelial cells of the eye, and especially before or in the absence of neovascularization. The therapeutic method involves administering a tetracycline, an analog of tetracycline, or a chemically modified tetracycline (CMT) to a patient suffering from such conditions. Also provided are compositions and methods for reducing breakdown of tight junctions in vascular endothelial cells; reducing IL-1 α concentrations; and inhibiting IL-1 α-mediated matrix metalloproteinase activity in endothelial cells of the eye and surrounding tissues.

### BACKGROUND OF THE INVENTION

Eye disorders and diseases such as age related macular degeneration (AMD) and diabetic retinopathy are the leading cause of acquired blindness in the developed world. The incidence of AMD is increasing as lifespan lengthens and the elderly population expands (D. S. Friedman et al., Arch. Ophthal. 122, 564 (2004)). All individuals with diabetes—both type 1 and type 2—are at risk for suffering from diabetic retinopathy, which manifests in non-proliferative and proliferative types.

Many retinal diseases are associated with ocular neovascularization, i.e., growth of new blood vessels in the cornea, retina or choroid causing a variety of subsequent pathologies resulting from bleeding, fibrosis and tissue damage. Proliferative diabetic retinopathy (PDR), for example, is associated with elevated expression of growth factors that promote pathogenic angiogenesis. In particular, vascular endothelial growth factor (VEGF) promotes new vessel formation in the diabetic retina and has been shown to be elevated in patients with PDR (Aiello et al., N. Engl. J. Med. 331: 1480 (1994)). VEGF is expressed in a variety of retinal tissues and further induces endothelial cell proliferation, favoring the formation of new vessels in the retina. In addition, basic fibroblast growth factor (bFGF) in the retina acts together with VEGF to induce formation of new vessels in which the subendothelial matrix has been shown to be weaker than in normal vessels. Certain proliferative retinal conditions and disorders manifest themselves before the onset of any ocular neovascularization. It would be useful to treat such conditions before the onset of neovascularization.

Other retinal conditions, disorders and diseases progress without measurable ocular neovascularization, such as those mainly characterized by leaking of existing blood vessels. The most prevalent retinal diseases that progress without neovascularization are non-proliferative diabetic retinopathy (NPDR), diabetic macular edema (DME), and cystoid macular edema (CME). Retinal vein occlusions are another condition in which ocular blood vessels can leak by themselves, although neovascularization can occur later on in this and other conditions. VEGF expression also facilitates a variety of other physiological changes in retinal tissue, apart from neovascularization, which promote fenestration of endothelial cells and associated fluid leakage, and which disrupts tight junctions between cells. The pro-inflammatory cytokine TNF-alpha has also been shown to play a role in diabetic retinopathy by altering endothelial cells in a way that may result in leaky barrier function and endothelial cell activation even absent or before any subsequent neovascularization.

Vascular and choroidal diseases are associated with conditions such as macular edema which lack substantial levels of neovascularization, and are also associated with conditions characterized by neovascularization in the eye. Fragile, abnormal blood vessels can develop and leak blood into the center of the eye and result in blurred vision. Macula edema can also occur when fluid leaks into the center of the macula, causing the macula to swell. Macular edema and retinopathy can be treated by laser surgery. Although focal laser treatment can stabilize vision, it can result in laser burns and the loss of side vision.

Leakage in retinal blood vessels and the growth of new blood vessels (and their subsequent leakage) can result from the activity of inflammatory cytokines and matrix metalloproteinases (MMPs) that facilitate the breakdown of vascular endothelial cell junctions. In the ocular epithelium, inflammatory and matrix degrading factors can be neutralized by treating with tetracycline and antimicrobial tetracycline analogs, and non-antimicrobial chemically modified tetracyclines, or “CMTs”. See, for example, U.S. Pat. No. 6,455,583 and U.S. Patent Publication No. 2003/0114426, incorporated herein by reference in its entirety. In particular, patients suffering from meibomian gland disease and/or ocular rosacea were found to have significantly greater concentrations of the pro-inflammatory cytokine interleukin 1-alpha (IL-1α) and markedly increased activity of one member of the MMP family, MMP-9 (also termed gelatinase B) in their tear fluid as compared to normal tears from asymptomatic patients. Topical administration of a tetracycline analog in an ointment (e.g., oxytetracycline) or in solution (e.g., doxycycline eye drops) lowered the concentrations of IL-1α, decreased pro-MMP9 activity and inhibited the maturation of inflammatory cytokine IL-1 beta in tear fluid, accompanied by complete resolution of symptoms in a majority of the treated patients.

It would be advantageous to identify compounds and compositions capable of inhibiting inflammatory and proteolytic activities in endothelial tissues, such as in the vascular endothelium of the eye. Such compounds and compositions would be useful, e.g., for inhibiting the breakdown of vascular endothelial cell junctions in the eye and would be beneficial for treating a variety of eye conditions, disorders and diseases characterized by breakdown of endothelial cells and tissues, including those which occur in the absence of any neovascularization, and those which occur at stages before substantial neovascularization has occurred.

### SUMMARY OF THE INVENTION

The present invention provides a class of agents (e.g., compounds and compositions comprising them), delivery systems comprising such agents, and methods for using such agents for treatment of a patient suffering from a condition associated with a retinal and/or choroidal disease or disorder of the eye involving endothelial cell dysfunction, especially endothelial cells of the vasculature. Such compositions and associated products and methods are useful for reducing or inhibiting pro-inflammatory cytokines and MMP activity in the endothelium of the eye and, therefore, reducing or inhibiting the breakdown of endothelial cell junctions and leakage in both new and pre-existing retinal blood vessels. Endothelial cell associated diseases, disorders and conditions of the eye include, without limitation, vascular leakage and choroidal neovascular disorders such as inflammatory macular edema, diabetic macular edema, cystoid macular edema, age related macular degeneration, retinitis pigmentosa, and retinopathy (proliferative and non-proliferative), such as diabetic retinopathy, sickle cell retinopathy and hypertensive retinopathy. Vascular leakage and neovascularization can also occur in disorders such as Central Retinal Vein Occlusion (CRVO) or Branch Retinal Vein Occlusion (BRVO).

The present invention also provides agents and methods for: i) inhibiting or reducing the breakdown of tight junctions in vascular endothelial cells; ii) reducing IL-1α concentration in eye tissues, including endothelial cells of the eye; and iii) inhibiting IL-1α-mediated matrix metalloproteinase activity in endothelial cells of the eye, which is increased in patients suffering from a vascular and/or choroidal disease or disorder of the eye. The present invention is thus useful for reducing or preventing the breakdown of endothelial cell junctions which thereby reduces leakage of retinal blood vessels (both new and pre-existing); reducing IL-1α concentration in endothelial cells of the eye and other tissues; and inhibiting MMP activity in endothelial cells of the eye and other tissues.

In certain embodiments, the present invention provides methods in which an effective amount of tetracycline or an antimicrobial tetracycline analog (referred to collectively as “antimicrobial tetracyclines”), or a non-antimicrobial analog of tetracycline is administered to a patient. Non-antimicrobial tetracycline analogs are commonly referred to and accepted in the scientific literature as “chemically modified tetracyclines” (CMTs). CMTs include tetracyclines which lack a dimethylamino side chain at position 4. Other examples of CMTs are described herein and elsewhere. Such tetracycline compounds, or compositions comprising those compounds, can be used for treating a vascular and/or choroidal disease or disorder of the eye, including those not associated with neovascularization. For vascular and/or choroidal diseases or disorders of the eye which are associated with neovascularization, such tetracycline compounds or compositions are advantageously administered before any signs of neovascularization are evident.

In one embodiment, the invention provides a method of treating a patient suffering from a condition associated with a retinal and/or choroidal disease or disorder of the eye involving endothelial cell dysfunction, especially endothelial cells of the vasculature, comprising administering to the patient an effective amount of an antimicrobial tetracycline compound or a composition comprising an antimicrobial tetracycline compound. In certain embodiments, the antimicrobial tetracycline compound or a composition comprising an antimicrobial tetracycline compound is administered to the patient in a non-antimicrobial amount. For vascular and/or choroidal diseases or disorders of the eye which are associated with neovascularization, the antimicrobial tetracycline compound or composition is advantageously administered before any signs of neovascularization are evident. Compounds and compositions useful in these methods are also provided.

In other embodiments, the invention provides a method of treating a patient suffering from a condition associated with a retinal and/or choroidal disease or disorder of the eye involving endothelial cell dysfunction, especially endothelial cells of the vasculature, comprising administering to the patient an effective amount of a non-antimicrobial tetracycline compound (CMT) or a composition comprising a CMT. For vascular and/or choroidal diseases or disorders of the eye which are associated with neovascularization, CMT compounds or compositions comprising a CMT compound are advantageously administered before any signs of neovascularization are evident. Compounds and compositions useful in these methods are also provided.

In certain embodiments, the methods of the invention comprise administering to the patient an effective amount of more than one tetracycline compound in combination, including combinations of more than one antimicrobial tetracycline, combinations of more than one non-antimicrobial tetracycline (CMT), or compositions comprising at least one CMT in combination with at least one antimicrobial tetracycline, in either a microbial or non-microbial amount.

In certain embodiments, the invention provides a method of treating a patient suffering from a condition associated with a retinal and/or choroidal disease or disorder of the eye involving endothelial cell dysfunction, especially endothelial cells of the vasculature, comprising administering a second therapeutic agent in combination with at least one antimicrobial tetracycline or CMT. In some embodiments, the second therapeutic agent is an anti-inflammatory agent. In other embodiments, the second therapeutic agent is a VEGF-inhibitor. For vascular and/or choroidal diseases or disorders of the eye which are associated with neovascularization, the compositions comprising one or more second therapeutic agent are advantageously administered before any signs of neovascularization are evident. Compositions useful in these methods are also provided.

In other embodiments, the methods and compositions of the invention inhibit one and preferably more than one of the following in endothelial cell junctions or blood vessels in the eye: a) matrix metalloproteinase activity; b) interleukin-1-alpha; c) synthesis and activation of interleukin-1β; and d) conversion of precursor interleukin-1β to mature interleukin-1β. In a specific embodiment, the matrix metalloproteinase is metalloproteinase-9. In another specific embodiment, the methods and compositions of the invention increase production of interleukin-1 receptor antagonist in the vascular endothelium or Bruch's Membrane of the eye.

### BRIEF DESCRIPTION OF THE DRAWINGS

FIGS. 1A-1C illustrate conditions associated with the breakdown of tight junctions in vascular endothelial cells. FIG. 1A shows vascular leakage associated with macular edema. FIG. 1B shows neovascularization in proliferative diabetic retinopathy. FIG. 1C shows choroidal neovascularization and vessel leakage associated with macular degeneration.

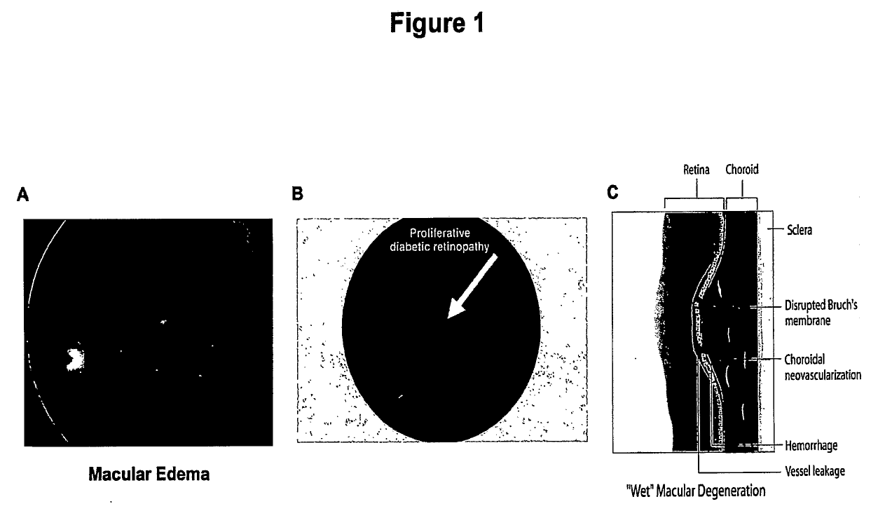

FIG. 2 is an illustration showing the breakdown of endothelial cell junctions which can be prevented by doxycycline treatment. Inflammatory cytokines and MMPs cause a breakdown of vascular endothelial tight junctions that leads to leakage in retinal blood vessels and/or growth of new blood vessels. Tetracycline, antimicrobial tetracycline analogs and CMTs can inhibit inflammation and MMP production, eliminating or reducing vascular changes.

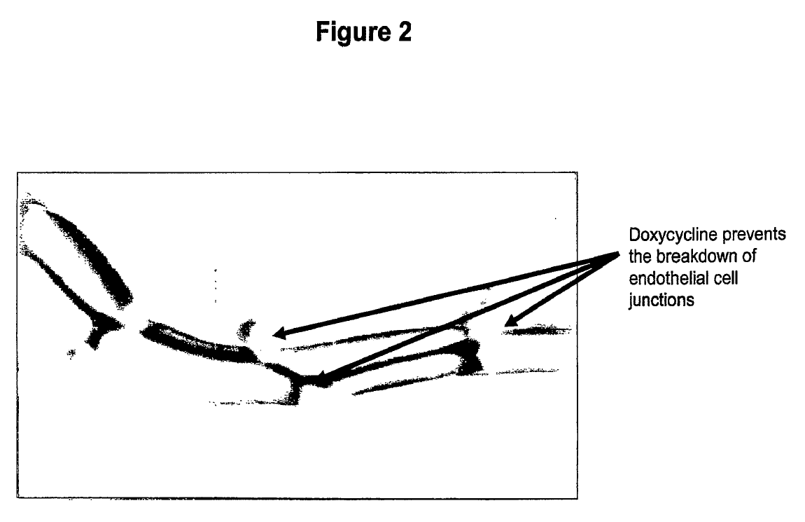

FIG. 3 illustrates routes of ocular administration, including intravitreal injection, intravitreal implant, and administration to the sub-conjunctival, juxtascleral, or sub-tenon's region of the eye.

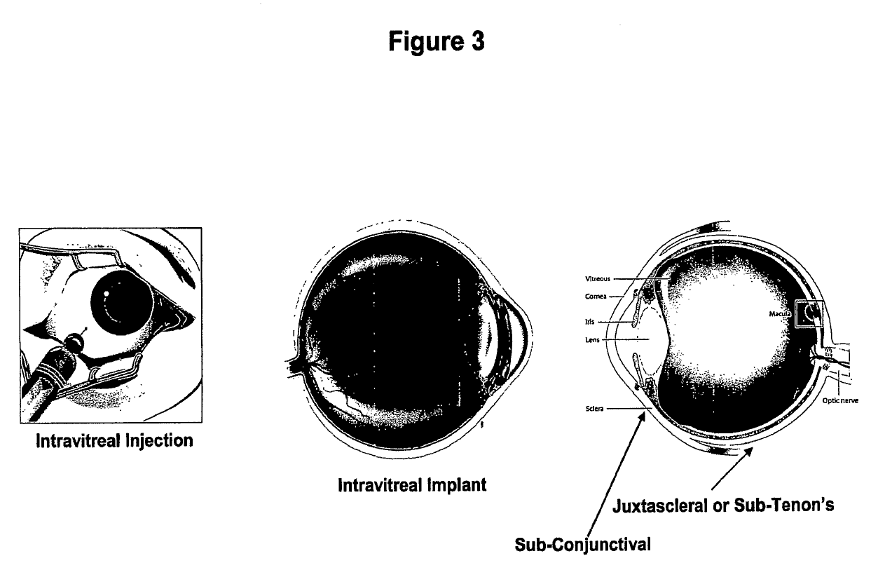

### DETAILED DESCRIPTION OF THE INVENTION

### Definitions

As used herein, tetracycline and its analogs having antimicrobial activity are termed “antimicrobial tetracyclines.” Antimicrobial tetracyclines may be administered in antimicrobial or sub-antimicrobial amounts.

As used herein, chemically modified tetracycline analogs that lack antimicrobial activity are termed “non-antimicrobial tetracyclines” or “chemically modified tetracyclines (CMTs).”

Unless otherwise specified, the term “tetracycline” is used herein to refer generically to tetracycline and tetracycline analogs, both antimicrobial and non-antimicrobial (CMTs).

As used herein, the term “patient” refers to an animal, preferably a mammal, and more preferably, a human.

### I. Methods of the Invention

The present invention provides methods using tetracycline compounds and compositions comprising them, including tetracycline, antimicrobial tetracycline analogs, and non-antimicrobial chemically-modified tetracyclines (CMTs), alone or in combination with each other and/or with other therapeutic agents, for the treatment of a patient suffering from a condition associated with a retinal and/or choroidal disease or disorder of the eye involving endothelial cell dysfunction, especially endothelial cells of the vasculature, and including relief of symptoms or conditions associated with the disease, disorder or condition. The methods of the present invention involve administering tetracycline compounds or compositions comprising one or more such compounds useful in reducing or inhibiting the breakdown of endothelial cell junctions by reducing or inhibiting inflammation and MMP activity, thus reducing or eliminating vascular changes that lead to vascular and/or choroidal diseases or disorders of the eye. In particular, antimicrobial tetracyclines and CMT compounds and compositions of the present invention are useful for treating or preventing vascular and/or choroidal diseases and disorders of the eye that are caused by the breakdown of cell junctions, such as vascular leakage and neovascular disorders.

Many retinal disorders or diseases are associated with pathologies resulting from ocular neovascularization. Of these, some are associated with retinal conditions and disorders that start before the onset of ocular neovascularization. Other retinal conditions, disorders and diseases progress without measurable ocular neovascularization, such as those mainly characterized by leaking of existing blood vessels. The main retinal diseases without evident or substantial levels of neovascularization include, without limitation: non-proliferative diabetic retinopathy, diabetic macular edema, and cystoid macular edema. Retinal vein occlusions are another condition in which ocular blood vessels can leak by themselves, although neovascularization can occur later on in this and other conditions.

Conditions associated with a vascular and/or choroidal disease or disorder of the eye include, without limitation, vascular leakage of the eye (with or without ocular neovascularization), choroidal disorders such as inflammatory macular edema, diabetic macular edema, cystoid macular edema, age related macular degeneration, retinitis pigmentosa, and retinopathy (proliferative and non-proliferative), such as diabetic retinopathy, sickle cell retinopathy and hypertensive retinopathy. Vascular leakage and neovascularization can also occur in disorders such as Central Retinal Vein Occlusion (CRVO) or Branch Retinal Vein Occlusion (BRVO).

In some embodiments, the methods and compositions of the invention inhibit vascular leakage of the eye before any neovascularization is evident. In other embodiments, the methods and compositions of the invention inhibit neovascularization in the eye. In other embodiments, the methods and compositions of the invention inhibit vascular leakage of the eye after neovascularization. In yet other embodiments, the methods and compositions of the invention inhibit choroidal neovascularization. In some embodiments, the compositions of the invention are administered in an amount effective to decrease inflammation or breakdown of endothelial cell junctions in the eye, blood vessels in the eye or cells of the retina, especially before any neovascularization is evident.

Antimicrobial and non-antimicrobial tetracycline compounds or compositions useful according to the methods of the invention may be administered topically, systemically, or locally, such as by intraocular injection (e.g., intravitreal injection), as part of a device or implant (e.g., a sustained release implant), or orally. Administration methods are discussed in more detail below.

In certain embodiments, the methods of the present invention comprise administering to a patient in need thereof one or more of tetracycline, an antimicrobial tetracycline analog or CMT compound or composition as the sole therapeutic agent(s). In certain other embodiments, the present invention provides methods in which one or more of the subject compounds (e.g., tetracycline, antimicrobial tetracycline analogs or CMTs) is administered to a patient in need thereof in combination with one or more additional therapeutic agents. Additional therapeutic agents that may be useful in the compositions and methods of the present invention include, without limitation: anti-inflammatory agents (e.g., steroids such as, for example, triamcinolone acetonide or TA, which has been used experimentally in AMD, CME and DME, corticosteroids, glucocorticoids, macrolide antibiotics and the like), non-steroidal anti-inflammatory agents (NSAIDs) (e.g., carprofen, flurbiprofen, ibuprofen, niflurnic acid, meclofenamic acid, ketoprofen, suxibutazone, diclofenac, mefenamic acid, tolfenamic acid, phenylbutazone and its metabolite oxyphenbutazone); metalloproteinase inhibitors (such as inhibitors to MMP-1, -2, -3, -7, -9, -13 and -14 which are present in eye tissues), immunosuppressive agents, anti-coagulants (e.g., low molecular weight heparin and various factors designed to promote blood coagulation), anti-angiogenic factors (e.g., various VEGF pathway inhibitors), retinoic acid derivatives (e.g., 9-cis-retinoic acid, 13-trans-retinoic acid and all-trans retinoic acid) vitamin D and its derivatives, estrogens, androgens, kinase inhibitors, growth factors, cytokines, vitamins and/or anti-oxidants.

Co-administration of the tetracycline and the additional therapeutic agent may, but need not be, at the same time. Co-administration may comprise treatment with different compositions or the therapeutic agents may be present in the same composition. Alternatively, co-administration includes administering the tetracycline and the additional therapeutic agent separately to the patient, such as at different times over the course of treatment, as long as each agent is present at the same time in the patient for at least a certain period. In some embodiments, compounds or compositions are administered to a patient in need thereof before any significant neovascularization, if any, is evident.

In certain embodiments of the invention, an additional therapeutic agent is an anti-inflammatory agent. Exemplary anti-inflammatory agents that may be used in conjunction with the methods and compositions of the invention include, without limitation: dexamethasone, prednisone, prednisolone, betamethasone, budesonide, cortisone, hydrocortisone, methylprednisolone, prednisone and triamcinolone, cyclosporine, tacrolimus, pimecrolimus, loteprednol, fluoromethalone, rimexolone, ketorolac, diclofenac, bromfenac and nepafenac. Other exemplary anti-inflammatory mediators or agents that may be used in conjunction with the methods and compositions of the invention include cytokines known to work antagonistically to a host of inflammatory mediators and pro-inflammatory cytokines known in the art including, without limitation, IL-1, IL-6, IL-12/23p40, CXCLi2, IFN-gamma, IL-20 and TNF-alpha and their cognate receptors. Anti-inflammatory mediators include, without limitation: TGF-beta 1, TGF-beta 4, prostaglandin E(2), and various known prostaglandin inhibitors, such as, for example, flurbiprofen, as well as other cyclooxygenase-2 inhibitors such as, for example, celecoxib, indomethacin, meloxicam, nabumetone, nimesulide and rofecoxib.

In other embodiments of the invention, an additional therapeutic agent is an agent that inhibits a member of the vascular endothelial growth factor (VEGF) family, a VEGF receptor (e.g., VEGFR 1 and 2), a protein in the VEGF pathway, or neuropilins, referred to collectively herein as VEGF-inhibitors or VEGF-inhibitory agents. Exemplary VEGF-inhibitory agents that may be used in conjunction with the methods and compositions of the invention are those that reduce or inhibit the activity of VEGF and related proteins include, for example, peptides, nucleic acids, antibodies, small molecules, and chemical compounds, including without limitation: pegaptinib (e.g., Macugen®), ranabizumab (e.g., Lucentis®), bevacizumab, VEGF-trap (e.g., by Regeneron, Inc.), anecortave acetate, or a tyrosine kinase inhibitor that inhibits VEGF activity. The chemokine stromal-derived factor 1 (SDF-1) stimulates VEGF expression and thus inhibitors of SDF-1 would be useful as VEGF inhibitor according to the present invention. Other VEGF-inhibitors may be similarly used.

In other embodiments of the invention, an additional therapeutic agent that may be used in conjunction with the methods and compositions of the invention is an agent, such as a nucleic acid, that regulates expression levels and/or the biological activity of a therapeutic agent involved in endothelial dysfunction. Such agents, include, for example, agents that mediate RNA interference (an “RNAi agent”), e.g., an siRNA, shRNA or miRNA, said agent comprising a nucleic acid or another delivery agent that encodes or delivers to a cell an agent capable of mediating RNAi thereby reducing the level of gene expression of a therapeutic agent involved in endothelial dysfunction, such as inflammatory molecules or VEGF pathway stimulatory agents. Agents that inhibit gene expression of one or more inflammatory molecules are anti-inflammatory agents useful in compositions and methods of the invention. Similarly, agents that inhibit gene expression of one or more VEGF pathway stimulatory agents may be VEGF inhibitors useful in compositions and methods of the invention.

In certain embodiments, an RNAi agent reduces or inhibits the activity of VEGF, VEGF receptors, or proteins in the VEGF pathway. For example, the present invention provides methods of treatment using compositions comprising a polynucleotide comprising an RNAi, siRNA, or miRNA sequence that acts through an RNAi or miRNA mechanism to attenuate expression of VEGF, a VEGF receptor, or a protein in the VEGF pathway. In one embodiment, the miRNA or siRNA sequence is between about 19 nucleotides and about 75 nucleotides in length, between about 21 and about 23 nucleotides in length, or between about 25 base pairs and about 35 base pairs in length. Such nucleic acid sequences may be single stranded or double stranded, with or without 5′ and/or 3′ overhangs and may comprise modified nucleoside bases and/or internucleotide linkages for increased stability and activity in vivo. In certain embodiments, the polynucleotide is a hairpin loop or stem-loop that may be processed by RNAse enzymes (e.g., Drosha and Dicer). Methods for generating and using siRNAs and miRNAs are well known in the art and can be found, for example, in Paddison et al. Proc. Natl. Acad. Sci. USA 2002, 99:1443-1448; Paddison et al. Genes & Dev. 2002, 16:948-958; Sui et al. Proc. Natl. Acad. Sci. USA 2002, 8:5515-5520; and Brummelkamp et al. Science 2002, 296:550-553. These reports describe methods to generate RNAs capable of specifically targeting numerous endogenously and exogenously expressed genes.

In certain embodiments of the invention, an additional therapeutic agent that may be used in conjunction with the methods and compositions of the invention is an aptamer that regulates the biological activity of a therapeutic agent involved in endothelial dysfunction. An “aptamer” is a nucleic acid molecule, such as RNA or DNA, that is capable of binding to a specific target molecule with high affinity and specificity (Ellington et al., Nature 346, 818-22 (1990); and Tuerk et al., Science 249, 505-10 (1990)). Such agents, include, for example, an aptamer that reduces or inhibits the activity of VEGF, VEGF receptors, or proteins in the VEGF pathway. Accordingly, one example of a therapeutic aptamer that may be administered in combination with tetracycline, an antimicrobial tetracycline analog, or CMT according to the invention is one that binds to and thereby modulates the activity of VEGF, VEGF receptors, or proteins in the VEGF pathway. Another example of a therapeutic aptamer that may be administered in combination with tetracycline, an antimicrobial tetracycline analog, or CMT according to the invention is one that binds to and thereby modulates the activity of compounds that play a role in inflammatory pathways, including proinflammatory cytokines and inflammatory mediators, such as, but on limited to, IL-1, IL-6, IL-12/23p40, CXCLi2, IFN-gamma, IL-20 and TNF-alpha; and their cognate receptors.

A tetracycline, an antimicrobial tetracycline analog, or CMT can be administered together (e.g., simultaneously) or at different times (e.g., sequentially) with one or more other agents, e.g., with one or more additional tetracyclines and/or with one or more other therapeutic agents. Combinatorial therapies may be achieved, for example, by contacting the damaged cells of the eye with a single composition or pharmacological formulation that includes both agents, or by contacting the cells with two distinct compositions or formulations at the same time. Alternatively, one agent may precede or follow administration of the other agent by intervals ranging from seconds, hours, days or weeks. In embodiments where two or more different kinds of therapeutic agents are applied separately to an individual, one would generally ensure that a significant period of time did not expire between the time of each delivery, such that these different kinds of agents would still be able to exert an advantageously combined effect on the target tissues or cells.

### II. Routes of Administration

In certain embodiments, methods of treating a patient suffering from a condition associated with a vascular and/or choroidal disease or disorder of the eye comprise administering a composition of the invention locally (e.g., by intraocular injection or insertion of a sustained release device that releases a composition of the invention), by topical means or by systemic administration (e.g., by routes of administration that allow in vivo systemic absorption or accumulation of drugs in the blood stream followed by distribution throughout the entire body, including, without limitation, by intravenous, subcutaneous, intraperitoneal, inhalation, oral, intrapulmonary and intramuscular routes). Intraocular administration of compositions of the invention includes, for example, delivery into the vitreous body, sub-conjunctival, juxtascleral, posterior scleral, and sub-tenon portions of the eye. See, for example, U.S. Pat. Nos. 6,943,145; 6,943,153; and 6,945,971. Tetracycline derivatives at doses in the range of about 1 pg/ml to about 2 mg/ml are substantially non-toxic when administered intraocularly. Generally, a substantially higher dose may be non-toxic when administered by topical or subconjunctival routes.

Tetracycline compounds (including antimicrobial analogs and CMTs) or compositions of the invention may be delivered by in a pharmaceutically acceptable ophthalmic formulation by intraocular injection. When administering the formulation by intravitreal injection, for example, the active agents should be concentrated so that minimized volumes may be delivered. Concentrations for injections may be at any amount that is effective and non-toxic, depending upon the factors described herein. In some embodiments, tetracycline compound(s) are formulated at doses of about 10 mg/ml or less, preferably 7.5 mg/ml or less, 6 mg/ml or less, 5 mg/ml or less, 4 mg/ml or less, 3 mg/ml or less, and are more preferably about 2 mg/ml or 1 mg/ml or less. In other embodiments, tetracycline compound(s) are formulated at doses of about 1 μg/ml to about 5 μg/ml; about 5 μg/ml to about 100 μg/ml; about 100 μg/ml to about 250 μg/ml; about 250 μg/ml to about 500 μg/ml; about 500 μg/ml to about 750 μg/ml; about 500 μg/ml up to 1 mg/ml; or more, as determined by the skilled practitioner. Tetracycline derivatives at doses of up to about 200 pg are substantially non-toxic when administered intravitreally.

Tetracycline compounds (including antimicrobial analogs and CMTs) or compositions of the invention may be delivered in a pharmaceutically acceptable ophthalmic vehicle, such that the composition is maintained in contact with the ocular surface for a sufficient time period to allow the compound to penetrate the affected regions of the eye, as for example, the anterior chamber, posterior chamber, vitreous body, aqueous humor, vitreous humor, cornea, iris/ciliary, lens, choroid, retina, sclera, suprachoroidal space, conjunctiva, subconjunctival space, episcleral space, intracorneal space, epicorneal space, pars plana, surgically-induced avascular regions, or the macula. Products and systems, such as delivery vehicles, comprising the agents of the invention, especially those formulated as pharmaceutical compositions—as well as kits comprising such delivery vehicles and/or systems—are also envisioned as being part of the present invention.

In certain embodiments, a therapeutic method of the invention includes the step of administering a tetracycline compound or composition of the invention by topical administration. In such embodiments, the concentration of tetracycline, antimicrobial tetracycline analog or CMT administered may depend upon the particular patient, the underlying disease and its severity, the dosing frequency, etc., as described herein and known to one skilled in the art. Sample concentrations include, but are not limited to, about 0.1 mg/ml to about 0.5 mg/ml, about 0.5 mg/ml to about 2.5 mg/ml, about 2.5 mg/ml to about 5 mg/ml, about 5 mg/ml to about 10 mg/ml, about 10 mg/ml to about 15 mg/ml, about 15 mg/ml to 30 mg/ml, or more, as determined by the skilled practitioner.

In certain embodiments, a therapeutic method of the invention includes the step of administering a tetracycline compound or composition of the invention as-an implant or device. In certain embodiments, the device is bioerodible implant for treating a medical condition of the eye comprising an active agent dispersed within a biodegradable polymer matrix, wherein at least about 75% of the particles of the active agent have a diameter of less than about 10 μm. The bioerodible implant is sized for implantation in an ocular region. The ocular region can be any one or more of the anterior chamber, the posterior chamber, the vitreous cavity, the choroid, the suprachoroidal space, the conjunctiva, the subconjunctival space, the episcleral space, the intracorneal space, the epicorneal space, the sclera, the pars plana, surgically-induced avascular regions, the macula, and the retina. The biodegradable polymer can be, for example, a poly(lactic-co-glycolic)acid (PLGA) copolymer. In certain embodiments, the ratio of lactic to glycolic acid monomers in the polymer is about 25/75, 40/60, 50/50, 60/40, 75/25 weight percentage, more preferably about 50/50. Additionally, the PLGA copolymer can be about 20, 30, 40, 50, 60, 70, 80 to about 90 percent by weight of the bioerodible implant. In certain preferred embodiments, the PLGA copolymer can be from about 30 to about 50 percent by weight, preferably about 40 percent by weight of the bioerodible implant.

Methods of administration may also be provided by rechargeable or biodegradable devices. Various slow release polymeric devices have been developed and tested in vivo in recent years for the controlled delivery of drugs, including proteinacious biopharmaceuticals. Accordingly, compositions of the invention may be delivered in time release capsules in a variety of carrier formulations such as in liposomes, microspheres, microcapsules, nanospheres, nanocapsules and the like. A variety of biocompatible polymers (including hydrogels), including both biodegradable and non-degradable polymers, can be used to form an implant for the sustained release of a drug at a particular target site.

Methods of introduction may additional be provided by non-biodegradable devices. In particular, compounds (e.g., tetracycline, tetracycline analogs, or CMTs) of the present invention can be administered via an implantable lens. The compound of the invention can be coated on the lens, dispersed throughout the lens or both. Additional description of implantable devices can be found, for example, in U.S. Publication Nos. 2004/0009222, 2004/0180075, 2005/0048099, 2005/0064010 and 2005/0025810, the contents of which are incorporated herein by reference.

### III. Tetracycline Compounds and Compositions Useful in the Methods of the Invention

The tetracycline utilized in the present invention may be any readily available, pharmaceutically acceptable tetracycline known in the medical art. Included in this group of tetracyclines are those such as chlortetracycline, which is marketed under the tradenames Acronize®, Aureocina®, Aureomycin®, Biomitsin®, Biomycin® and Chrysomykine®; demeclocycline marketed as Ledermycin®, Detravis®, Meciclin®, and Mexocine®; doxycyline marketed as Vibramycin®, Vibramycin®, Hyclace®, Liomycin®, Vibradox®, Panamycin®, Titradox®, Hydramycin® and Tecacin®; lymecycline which is marketed as Armyl®, Mucomycin®, Tetramyl® and Tetralysal®; methacycline which is marketed as Adriamicina®, Cyclobiotic®, Germicilclin®, Globociclina®, Megamycine®, Pindex® and Londomycin®; Optimycin®, Rondomycin®; minocycline which is marketed as Minocin®, Klinomycin® and Vectrin®; oxytetracycline which is marketed as Biostat®, Oxacycline®, Oxatets®, Oxydon®, Oxymycin®, Oxytan®, Oxytetracid®, Ryomycin®, Stezazin®, Tetraject®, Terramycin®, Tetramel®, Tetran®, Dendarcin® and Dendarcin®; rolitetracycline marketed as Bristacin®, Reverin®, Superciclin®, Syntetrex®, Syntetrin®, Synotodecin®, Tetraverin®, Transcycline®, Velacicline® and Velacycline®; and tetracycline marketed as Achromycin®, Ambramycin®, Cyclomycin®, Polycycline®, Tetrabon® and Tetracyn®.

Active salts of tetracycline which are formed through protonation of the dimethylamino group on carbon atom 4, exist as crystalline compounds and are very stable in water. However, these amphoteric antibiotics will crystallize out of aqueous solutions of their salts unless stabilized by an excess of acid. The hydrochloride salts are used most commonly for oral administration. Water soluble salts may be obtained also from bases such as sodium or potassium hydroxides but are not stable in aqueous solution, they are also formed with divalent and polyvalent metals.

When used in sub- or non-antimicrobial amounts, tetracycline and antimicrobial tetracycline analogs used according to the present invention may be administered at a dosage level from about 10% to about 100%, and preferably about 20% to about 80% of the normal antibiotic therapeutic dose of the particular tetracycline compound being employed. By normal antibiotic therapeutic dose is meant the dosage of the particular tetracycline compound which is commonly used and recommended for the treatment of bacterial infection. Alternatively, sub-antimicrobial dose means a dose having no significant antimicrobial effect in vitro or in vivo. More than 100% of the normal antibiotic therapeutic dose may be utilized in methods of the present invention. The normal antibiotic therapeutic dose of tetracyclines is well studied and well documented and may be determined empirically for specific patient treatment regimens.

In certain embodiments, the compounds useful according to the present invention are tetracyclines that have been chemically modified so as to substantially reduce or eliminate antimicrobial properties and increase their antimicrobial-inflammatory activity. Methods for reducing antimicrobial properties of a tetracycline are disclosed in The Chemistry of the Tetracyclines, Ch. 6, Mitscher, Ed., at p. 211. As pointed out by Mitscher, modification of tetracycline at positions 1, 2, 3, 4, 10, and 12a can lead to loss of antimicrobial activity. Such chemically modified tetracyclines (CMTs) are included in certain embodiments of the present invention because they can be used without disturbing the normal microbial flora of the treated subject as would happen with extended exposure to antimicrobial tetracyclines.

CMTs are useful in patients who are unable to tolerate tetracyclines for extended periods of time. The intolerance to tetracyclines can manifest itself in gastrointestinal problems, e.g., epigastric pain, nausea, vomiting, and diarrhea, or other problems related to taking long-term oral antibiotics. CMTs (or locally applied tetracyclines) can have greater efficacy because of the higher concentrations that can be achieved at the disease site. Because of their lack of antimicrobial-bacterial effect and greater therapeutic activity, CMTs can have fewer systemic or other side effects than tetracyclines, whether administered, e.g., by intraocular injection, orally or topically.

Preferred CMTs used according to the present invention include those lacking a dimethylamino side chain at position 4. For example, 4-dedimethylamino-tetracycline, 4-dedimethylamino-5-oxytetracycline, 4-dedimethylamino-7-chlortetracycline, 4-hydroxy-4-dedimethylaminotetracycline, 4-dedimethylamino-12a-deoxytetracycline, 4-dedimethylamino-11-hydroxy-12a-deoxytetracycline, 4-dedimethylamino-7-dimethylaminotetracycline, 6-dimethyl-6-deoxy-4-dedimethylaminotetracycline, 6-O-deoxy-5-hydroxy-4-dedimethylaminotetracycline, 11a-chlortetracycline, 12a-deoxytetracycline, and the 2-nitrilo analogs of tetracycline.

The amount of tetracycline, antimicrobial tetracycline analog, or CMT administered to effectively treat a condition associated with a vascular and/or choroidal disease or disorder of the eye, is an amount that significantly decreases or inhibits one or more of: chroidal neovascularization; inflammation or breakdown of endothelial cell junctions in the eye, blood vessels in the eye or cells of the retina; matrix metalloproteinase (e.g., matrix metalloproteinase 9) activity in endothelial cell junctions or blood vessels in the eye; interleukin-1-alpha; synthesis and activation of interleukin-1β; or conversion of precursor interleukin-1 to mature interleukin-1β. In another embodiment, the amount of tetracycline, antimicrobial tetracycline analog, or CMT administered is effective to increase production of interleukin-1 receptor antagonist in the vascular endothelium or Bruch's Membrane of the eye. The maximal dosage for humans is the highest dosage that does not cause clinically important side effects. For the purpose of the present invention, side effects include clinically important disruption of the normal flora as well as harmful or toxic effects to the eye surface and/or retinal surface.

The dosage of tetracycline(s) administered in accordance with the present invention is also additionally dependent upon the age and weight of the person being treated, the mode of administration, and the type and severity of the inflammatory or matrix-degrading disease being treated. It is understood that the dosage regimen can be determined by a skilled artisan, such as a physician, considering various factors that modify the action of the compounds and compositions of the invention. For illustrative purposes, a suitable amount of the antimicrobial tetracycline analog doxycycline is 0.1-4.0 mg/kg/day. In the case of a non-antimicrobial tetracycline, for example, the dose for 4-dedimethylaminotetracycline can be 0.1-30 mg/kg/day.

The volume of composition administered according to the methods described herein is also dependent on factors such as the mode of administration, quantity of the tetracycline, tetracycline analog, or CMT administered, age and weight of the patient, and type and severity of the inflammatory or matrix-degrading disease being treated. For example, if administered orally as a liquid, the liquid volume comprising a composition of the invention may be from about 0.5 milliliters to about 2.0 milliliters, from about 2.0 milliliters to about 5.0 milliliters, from about 5.0 milliliters to about 10.0 milliliters, or from about 10.0 milliliters to about 50.0 milliliters. If administered by injection, the liquid volume comprising a composition of the invention may be from about 5.0 microliters to about 50 microliters, from about 50 microliters to about 250 microliters, from about 250 microliters to about 1 milliliter, from about 1 milliliter to about 5 milliliters, from about 5 milliliters to about 25 milliliters, from about 25 milliliters to about 100 milliliters, or from about 100 milliliters to about 1 liter.

In certain embodiments, tetracycline, antimicrobial tetracycline analogs or CMTs for topical, systemic or local administration can be administered in a range from about 0.001% to about 3.0% (weight per volume or weight per weight), or from about 0.001% to about 0.01%, from about 0.01% to about 0.025%, from about 0.025% to about 0.05%, from about 0.05% to about 0.1%, from about 0.1% to about 0.25%, from about 0.25% to about 1.0%, from about 1.0% to about 2.0%, or from about 2.0% to greater than 3.0%, i.e., about 3.0% to about 10.0% or greater. In other embodiments, CMTs for topical, systemic or local administration can be administered in a range from 0.001% to 10%, or from about 0.001% to about 0.1%, from about 0.1% to about 1.0%, from about 1.0% to about 2.5%, from about 2.5% to about 5.0%, or from about 5.0% to greater than 10.0%, i.e., about 10.0% to about 20.0% or greater.

If administered topically, tetracycline, antimicrobial tetracycline analogs or CMT preparations can be administered to the preocular tear film or onto the eyelid skin or lid margin 1 to 6 times per day for a period of 1-4 weeks, 1-3 months, 3-6 months, 6-12 months, 1-2 years, or more, up to the lifetime of the patient. For example, an eye drop solution comprising doxycycline as an active ingredient can be prepared by dissolving pharmaceutical grade doxycycline hydrochloride powder in an electrolyte-balanced salt solution (BSS™, Alcon, Ft. Worth, Tex.) to a final concentration of 0.025%.

If administered by intraocular injection, tetracycline, antimicrobial tetracycline analogs or CMT compositions can be delivered one or more times periodically throughout the life of a patient. For example, tetracycline, an antimicrobial tetracycline analog or CMT composition can be delivered once per year, once every 6-12 months, once every 3-6 months, once every 1-3 months, or once every 1-4 weeks. Alternatively, more frequent administration may be desirable for certain conditions or disorders. If administered by an implant or device, tetracycline, antimicrobial tetracycline analog, or CMT compositions can be administered one time, or one or more times periodically throughout the lifetime of the patient, as necessary for the particular patient and disorder or condition being treated.

The dosage of agents administered in combination with a tetracycline, an antimicrobial tetracycline analog, or CMT according to the present invention is dependent upon the age and weight of the patient being treated, the mode of administration, interactions between one or more compounds included in the composition (i.e., inhibitory, additive or synergistic) and the type and severity of the inflammatory or matrix-degrading disease being treated. Such factors are readily understood by the skilled practitioner.

For illustrative purposes, a suitable amount of a VEGF-inhibitor, is 0.1-4.0 mg/kg/day, depending on the inhibitor, its formulation and the patient's individual needs. When the VEGF-inhibitor is Lucentis® (a humanized, anti-VEGF antibody fragment), it should typically be administered in either about 300 or about 500 microgram doses in multiple doses. When the VEGF-inhibitor is Macugen®, it should typically be administered in a dose ranging from either about 0.3 mg to about 3.0 mg periodically, e.g., once every 2, 3, 4, 5, 6, 8 or more weeks.

In general, an anti-inflammatory agent or VEGF-inhibitor is administered in a quantity of about 50 micrograms to about 800 milligrams, about 100 micrograms to about 200 milligrams, or about 500 micrograms to about 100 milligrams. In other embodiments, an anti-inflammatory agent or VEGF-inhibitor is administered in a quantity of about 100 micrograms to about 1 milligram, about 1 milligram to about 5 milligrams, about 5 milligrams to about 25 milligrams, about 25 milligrams to about 250 milligrams, about 250 milligrams, or about 250 milligrams to about 1 gram. In a preferred embodiment, an anti-inflammatory agent or a VEGF-inhibitor has a final concentration of about 100 micrograms to about 2 milligrams or about 10 micrograms to about 1 gram.

In certain embodiments, compounds (e.g., tetracycline, antimicrobial tetracycline analogs, or CMTs) of the present invention are formulated with a pharmaceutically acceptable carrier. For example, any of the above tetracyclines may be administered alone or as a component of a pharmaceutical formulation. The subject compounds may be formulated for administration in any convenient way for use in human or veterinary medicine.

In certain embodiments, pharmaceutical compositions suitable for parenteral administration may comprise tetracycline, an antimicrobial tetracycline analog or CMT in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The compositions of the invention may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like in the compositions. In addition, prolonged absorption of the injectable pharmaceutical form may be brought about by the inclusion of one or more agents that delay absorption, such as, e.g., aluminum monostearate and gelatin.

When administered, the therapeutic composition for use in this invention is, of course, in a pyrogen-free, physiologically acceptable form. Further, the composition may desirably be encapsulated or injected in a viscous form into the vitreous humor for delivery to the site of retinal or choroidal damage. In addition to tetracycline, antimicrobial tetracycline analogs, or CMTs, one or more additional therapeutically useful agents, such as anti-inflammatory or VEGF-inhibiting agents, may optionally be included in any of the compositions, as described above. Such additional therapeutic agents include, without limitation: anti-inflammatory agents (e.g., steroids such as, for example, triamcinolone acetonide or TA, which has been used experimentally in AMD, CME and DME, corticosteroids, glucocorticoids, macrolide antibiotics and the like), non-steroidal anti-inflammatory agents (NSAIDs) (e.g., carprofen, flurbiprofen, ibuprofen, niflumic acid, meclofenamic acid, ketoprofen, suxibutazone, diclofenac, mefenamic acid, tolfenamic acid, phenylbutazone and its metabolite oxyphenbutazone); metalloproteinase inhibitors (such as inhibitors to MMP-1, -2, -3, -7, -9, -13 and -14 which are present in eye tissues), immunosuppressive agents, anti-coagulants (e.g., low molecular weight heparin and various factors designed to promote blood coagulation), anti-angiogenic factors (e.g., various VEGF pathway inhibitors), retinoic acid derivatives (e.g., 9-cis-retinoic acid, 13-trans-retinoic acid and all-trans retinoic acid) vitamin D and its derivatives, estrogens, androgens, kinase inhibitors, growth factors, cytokines, vitamins and/or anti-oxidants.

Certain compositions disclosed herein may be administered topically, either to skin or to mucosal membranes. The topical formulations may further include one or more of the wide variety of agents known to be effective as skin or stratum corneum penetration enhancers. Examples of these include, without limitation: 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur.

Dosage forms for the topical or transdermal administration include powders, sprays, ointments, pastes, creams, lotions, gels, solutions, patches, and inhalants. The active compound may be mixed under sterile conditions with a pharmaceutically acceptable carrier, and with any preservatives, buffers, or propellants which may be required. The ointments, pastes, creams and gels may contain, in addition to a subject compound of the invention (e.g., tetracycline, a antimicrobial tetracycline analog, or a CMT), excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a subject compound, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

In certain embodiments, methods of the invention can be administered orally, e.g., in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent as an active ingredient. An agent may also be administered as a bolus, electuary or paste.

In solid dosage forms for oral administration (capsules, tablets, pills, dragees, powders, granules, and the like), one or more therapeutic compounds of the present invention may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

### EXAMPLES

The following Examples detail compositions and methods illustrative of the present invention. It will be apparent to those skilled in the art that many modifications, both of materials and methods, may be practiced without departing from the purpose and intent of this disclosure.

All animal study protocols used herein complied with the Biological Test Center's animal welfare policies and were reviewed and approved by the Institutional Animal Care and Use Committee.

### Example 1

### Doxycycline Reduces VEGF-Mediated Vascular Damage

Retinal vascular leakage was induced in rabbits by intravitreal injection of VEGF according to the procedures set forth in Edelman et al., Experimental Eye Research 80:249-258 (2005). The protective effect of doxycycline on VEGF-mediated retinal vascular damage was assessed. A total of 12 Dutch Belted rabbits (Covance) were anesthetized with isoflurane inhalation and topical 0.5% proparacaine hydrochloride and each eye studied (24 eyes total). Four animals received a 50 μl intravitreal injection of 0.025% bovine serum albumin (BSA) in sterile phosphate-buffered saline (PBS) (Dow Pharmaceutical Sciences) (vehicle, eight eyes total). Doxycycline was formulated from anhydrous doxycycline (Dow Pharmaceutical Sciences) and reconstituted as a saturated solution of doxycycline in 0.025% BSA in PBS assayed to be at a final doxycycline concentration of approximately 0.067 weight percent. Four animals received 50 μl of the doxycycline formulation in each eye (eight eyes total). Four animals received 50 μl of a 40 mg/mL solution of triamcinolone acetonide (Kenalog®, Bristol Myers-Squibb) in 0.025% BSA in PBS (positive control, eight eyes total). Each injection was followed by a 50 μl intravitreal injection of a 10 μg/mL solution of recombinant human VEGF₁₆₅ (Vascular Endothelial Growth Factor 165 amino acid residue variant, R&D Systems) in 0.025% BSA in PBS. A 30-gauge ⅝-inch needle was used for the injections. VEGF was purchased from R&D Systems (Minneapolis, Minn.).

Prior to injections, eyes were prepared for injection with 1% tropicamide (2 drops), followed ten minutes later by phenylephrine hydrochloride 2.5% (2 drops). An ophthalmic Betadine solution was then used to moisten the eyes. Eyes were moistened with an ophthalmic Betadine solution. After five minutes, the Betadine was washed out of the eyes with sterile saline and proparacaine hydrochloride was delivered to each eye.

Animals were then anesthetized with an intravenous injection of a ketamine/xylazine cocktail (77 mg/mL ketamine, 23 mg/mL xylazine) at 0.1 mL/kg.

Vascular leakage, hemorrhage, and edema were assessed using fundus photography and fluorescein angiography (see Table 1). Rabbits were anesthetized with subcutaneous 100 mg/mL ketamine at 35 mg/kg and 100 mg/mL xylazine at 7 mg/kg, eyes were dilated with topical 1% tropicamide, and angiograms were obtained with a Topeon TRC-501X retinal camera coupled to a personal computer with IMAGEnet 2000 software (Topcon Medical Systems Inc., Paramus, N.J.). Intravenous sodium fluorescein injection (12 mg/kg) was performed 48 hours after intravitreal injection of VEGF. Late-phase angiography was performed 4-6 min after intravenous sodium fluorescein injection,

TABLE 1
Mean Scores of Efficacy Parameters at 48 Hours Post-Dosing

| | | | | Difference | Difference | Difference |
|---|---|---|---|---|---|---|
| | Vehicle | Doxy | Kenalog | (Doxy-Vehicle) | (Kenalog-Vehicle) | (Kenalog-Doxy) |
| Left Eye | | | | | | |
| Leakage | 2.75 | 3.13 | 0.50 | 0.38 | −2.25 | −2.63 |
| Hemorrhage/Microaneurism | 2.25 | 2.25 | 1.13 | 0.00 | −1.13 | −1.13 |
| Edema | 2.75 | 3.25 | 1.25 | 0.50 | −1.50 | −2.00 |
| Right Eye | | | | | | |
| Leakage | 2.25 | 1.75 | 0.25 | −0.50 | −2.00 | −1.50 |
| Hemorrhage/Microaneurism | 2.25 | 2 | 0.75 | −0.25 | −1.50 | −1.25 |
| Edema | 2 | 1.75 | 1.13 | −0.25 | −0.88 | −0.63 |
| Combined Eyes | | | | | | |
| Leakage | 2.81 | 2.06 | 0.25 | −0.75 | −2.56 | −1.81 |
| Hemorrhage/Microaneurism | 2.56 | 1.63 | 1.00 | −0.94 | −1.56 | −0.63 |
| Edema | 2.69 | 2.00 | 1.13 | −0.69 | −1.56 | −0.88 |

The greatest amount of VEGF-mediated retinal vascular damage was found in the vehicle treated group. Leakage (left eye: 2.75 units; right eye: 2.25 units; and, combined eyes: 2.81 units), hemorrhage/microaneurism (left eye: 2.25 units; right eye: 2.25 units; and, combined eyes: 2.81 units), and edema (left eye: 2.75 units; right eye: 2.0 units; and, combined eyes: 2.69 units) were highest in this group. Unexpectedly, VEGF-mediated retinal vascular damage was reduced in the doxycycline-treated group as compared to the rabbits that received vehicle. Leakage in the combined eyes was (left eye: 3.13 units; right eye: 1.75 units; and, combined eyes: 2.06 units). Hemorrhage/microaneurism was also lower (left eye: 2.25 units; right eye: 2.0 units; and, combined eyes: 1.63 units). Edema was also lower (left eye: 3.25 units; right eye: 1.75 units; and, combined eyes: 2.0 units).

As expected, Kenalog® treated animals exhibited very little response to VEGF-mediated retinal vascular damage. Leakage was sharply lower as compared to the rabbits receiving vehicle (left eye: 0.50 units; right eye: 0.25 units; and, combined eyes: 0.25 units). Hemorrhage/microaneurism was also lower compared to rabbits receiving vehicle (left eye: 1.13 units; right eye: 0.75 units; and, combined eyes: 1.00 units). Edema was also found to be lower as compared to rabbits receiving vehicle (left eye: 1.25 units; right eye: 1.13 units; and, combined eyes: 1.13 units).

### Example 2

### Oral Doxycycline Treatment to Inhibit Leakage

In the Vascular Endothelium in Humans

Ten patients suffering from diabetic retinopathy are treated with oral doxycycline 50 mg orally twice a day for eight weeks. The patients are monitored for improvement of macular edema and vascular leakage by fluorescein angiography and scanning ocular fluorophotometry. Decreased vascular leakage in response to doxycycline administration is observed, which leads to a decrease in edema and improved visual acuity.

### Example 3

### Topical Doxycycline Treatment to Inhibit Leakage

In the Vascular Endothelium in Humans

Ten patients suffering from diabetic retinopathy are treated with topical doxycycline. Two patients each are administered doxycycline at a concentration selected to be between 0.025% and 0.1% daily for one month. The patients are monitored for improvement of macular edema and vascular leakage by fluorescein angiography and scanning ocular fluorophotometry. Decreased vascular leakage in response to doxycycline administration is observed, which leads to a decrease in edema and improved visual acuity in most or all of the patients treated.

### Example 4

### Doxycycline Treatment to Reduce MMP-9 Activity

In Human Corneal Epithelial Cultures

Independent human choroidal cultures are exposed to various concentrations of doxycycline (0.001%, 0.01%, 0.025%, 0.05%, 0.1%, 0.25%, 0.5%, 1.0%, 1.5%, 2.0%, 2.5%, 3.0%, and 5.0%) for 0.5 hours, 1 hour, 2 hours, 4 hours, 6 hours, 8 hours, 12 hours, 24 hours, 36 hours, or 48 hours. MMP-9 (gelatinase) activity is evaluated by gelatin zymography. Gelatin zymography can be performed under nonreducing conditions on 7.5% polyacrylamide mini slab gels (Bio-Rad), copolymerized with 1.5 g/L 90 Bloom gelatin (Sigma). Aliquots containing 50 μg of total protein is used for each zymographic test. One part sample is mixed with one part Tris-Glycine SDS Sample Buffer (2×) and kept at room temperature for 10 minutes. Samples are applied to the polyacrylamide gel and subject to electrophoresis according to standard conditions for 60-120 minutes. After electrophoresis, the gel is incubated for 30 minutes at room temperature in zymogram renaturing buffer. The renaturing buffer is decanted and replaced with zymogram developing buffer for 30 minutes at room temperature. The gel is incubated in fresh zymogram developing buffer for an additional four hours at 37° C. The gel is then stained with 0.5% Coomassie Blue R-250 for 30 minutes and destained with Coomassie R-250 destaining solution (Methanol:Acetic acid:Water; 50:10:40).

The level of pro-MMP9 activity in the supernatant is expected to decrease with increasing concentrations of doxycycline compared to untreated cultures. MMP-9 is the metalloproteinase responsible for breakdown of endothelial cell junctions and is also capable of cleaving precursor IL-1β into its mature form. Similarly, pre-IL-1β cleavage is monitored by ELISA and is shown to correlate directly with increasing doxycycline concentrations.

All cited references are hereby incorporated herein by reference in their entirety.

What is claimed is:

1. A method of treating a patient suffering from a condition associated with a retinal and/or choroidal disease or disorder of the eye involving endothelial cell dysfunction without evident or substantial levels of ocular neovascularization, said method comprising administering an effective amount of a tetracycline to said patient.

2. A method of treating a patient suffering from a condition associated with a retinal and/or choroidal disease or disorder of the eye involving endothelial cell dysfunction without evident or substantial levels of ocular neovascularization, said method comprising administering an effective amount of a non-antimicrobial tetracycline to said patient.

3. The method of claim 1 or 2, wherein the endothelial cells are vascular cells.

4. The method of claim 1 or 2, wherein said treatment comprises inhibiting vascular leakage in the eye.

5. The method of claim 1 or 2, wherein said amount decreases inflammation of endothelial cell junctions in the eye, blood vessels in the eye or cells of the retina.

6. The method of claim 1 or 2, wherein said amount reduces breakdown of cell junctions in the endothelium of the eye, blood vessels in the eye or in the retina.

7. The method of claim 1 or 2, wherein said condition is selected from the group consisting of vascular leakage in the eye, macular edema, non-proliferative diabetic retinopathy, and central or branch retinal vein occlusion.

8. The method of claim 7, wherein said macular edema is selected from the group consisting of inflammatory macular edema, diabetic macular edema, and cystoid macular edema.

9. The method of claim 1, wherein said tetracycline is an antimicrobial tetracycline analog.

10. The method of claim 9, wherein the tetracycline analog is selected from oxytetracycline, doxycycline and minocycline.

11. The method of claim 10, wherein said tetracycline is doxycycline.

12. The method of claim 1, wherein said amount of tetracycline is non-antimicrobial.

13. The method of claim 2, wherein said non-antimicrobial tetracycline is a tetracycline which lacks a dimethylamino side chain at position 4.

14. The method of claim 2, wherein said non-antimicrobial tetracycline is a member selected from the group consisting of: 4-dedimethylaminotetracycline, 4-dedimethylamino-5-oxytetracycline, 4-dedimethylamino-7-chlorotetracycline, 4-hydroxy-4-dedimethylaminotetracycline, 4-dedimethylamino-12a-deoxytetracycline, 4-dedimethylamino-11-hydroxy-12a-deoxytetracycline, 4-dedimethylamino-7-dimethylaminotetracycline, 6-dimethyl-6-deoxy-4-dedimethylaminotetracycline, 6-o-deoxy-5-hydroxy-4-dedimethylaminotetracycline, 11a-chlortetracycline, 12a-deoxytetracycline and 2-nitrilo analogs of tetracycline.

15. The method of claim 1 or 2, further comprising administering to said patient an effective amount of a second therapeutic agent.

16. The method of claim 15, wherein the second therapeutic agent is selected from the group consisting of an anti-inflammatory agent, an immunomodulatory agent, a growth factor, a cytokine and a VEGF-inhibitor.

17. The method of claim 15, wherein the second therapeutic agent is an anti-inflammatory corticosteroid.

18. The method of claim 15, wherein the second therapeutic agent is an anti-inflammatory agent selected from the group consisting of: dexamethasone, prednisolone, betamethasone, budesonide, cortisone, hydrocortisone, methylprednisolone, prednisone and triamcinolone, cyclosporine, tacrolimus, pimecrolimus, loteprednol, fluoromethalone, rimexolone, ketorolac, diclofenac, bromfenac and nepafenac.

19. The method of claim 15, wherein said second therapeutic agent is a VEGF-inhibitor.

20. The method of claim 19, wherein said VEGF-inhibitor is egaptinib, ranabizumab, bevacizumab, a VEGF-trap, anecortave acetate, or a tyrosine kinase inhibitor.

21. The method of claim 1 or 2, wherein said treatment comprises inhibiting one or more of: matrix metalloproteinase activity in endothelial cell junctions or blood vessels in the eye, interleukin-1-alpha, synthesis and activation of interleukin-1β, and conversion of precursor interleukin-1β, to mature interleukin-1β.

22. The method of claim 21, wherein said treatment inhibits matrix metalloproteinase-9.

23. The method of claim 1 or 2, wherein said treatment comprises increasing production of interleukin-1 receptor antagonist in the vascular endothelium or Bruch's Membrane of the eye.

24. The method of claim 1 or 2, wherein said tetracycline is topically administered to an eye of said patient suffering from a condition associated with retinal and/or choroidal vascular disease without evident or substantial levels of ocular neovascularization.

25. The method of claim 1 or 2, wherein said tetracycline is orally administered to said patient suffering from a condition associated with retinal and/or choroidal vascular disease without evident or substantial levels of ocular neovascularization.

26. The method of claim 1 or 2, wherein said tetracycline is administered by intraocular injection to the eye of said patient suffering from a condition associated with retinal and/or choroidal vascular disease without evident or substantial levels of ocular neovascularization.

27. The method of claim 26, wherein said injection delivers said tetracycline into the vitreous body of the eye of said patient suffering from a condition associated with retinal and/or vascular disease without evident or substantial levels of ocular neovascularization.

28. The method of claim 26, wherein said injection delivers said tetracycline to the posterior sclera of the eye of said patient suffering from a condition associated with retinal and/or choroidal vascular disease without evident or substantial levels of ocular neovascularization.

29. The method of claim 26, wherein said injection delivers said tetracycline to the sub-conjunctiva of the eye of said patient suffering from a condition associated with retinal and/or choroidal vascular disease without evident or substantial levels of ocular neovascularization.

30. The method of claim 1 or 2, wherein said tetracycline is administered by an intravitreal implant to the eye of said patient suffering from a condition associated with retinal and/or choroidal vascular disease without evident or substantial levels of ocular neovascularization.

31. The method of claim 30, wherein said implant is a sustained release implant.

32. A composition comprising an anti-inflammatory agent, an amount of a tetracycline effective for treating a patient suffering from a condition associated with a retinal and/or choroidal vascular disease or disorder without evident or substantial levels of ocular neovascularization, and a pharmaceutically acceptable carrier.

33. The composition of claim 32, wherein said anti-inflammatory agent is selected from the group consisting of: dexamethasone, cyclosporine, tacrolimus, pimecrolimus, prednisolone, loteprednol, fluoromethalone, rimexolone, ketorolac, diclofenac, bromfenac and nepafenac.

34. The composition of claim 32, wherein said anti-inflammatory agent has a final concentration of between about 100 micrograms to about 2 milligrams.

35. The composition of any one of claims 32-34, wherein said tetracycline has a final concentration of between about 0.001% to about 3.0%.

36. A composition comprising a VEGF-inhibitor, an amount of a tetracycline effective for treating a patient suffering from a condition associated with a retinal and/or choroidal vascular disease or disorder without evident or substantial levels of ocular neovascularization, and a pharmaceutically acceptable carrier.

37. The composition of claim 36, wherein said VEGF-inhibitor is selected from the group consisting of: egaptinib, ranabizumab, bevacizumab, a VEGF-trap, anecortave acetate, and a tyrosine kinase inhibitor.

38. The composition of claim 36, wherein said VEGF-inhibitor has a final concentration of between about 100 micrograms to about 2 milligrams.

39. The composition of claim 36, wherein said tetracycline has a final concentration of between about 0.001% to about 3.0%.
